(19) United States
(12) Patent Application Publication
(10) Pub. No.: US 2018/0050172 A1
(43) Pub. Date: Feb. 22, 2018

(54) CONTINUOUS BLADDER IRRIGATION ADAPTOR FOR A FOLEY CATHETER

(71) Applicant: John Farley, Pompano Beach, FL (US)

(72) Inventor: John Farley, Pompano Beach, FL (US)

(21) Appl. No.: 15/242,669

(22) Filed: Aug. 22, 2016

(51) Int. Cl. A61M 25/00; A61M 3/02; A61M 39/10

(52) U.S. Cl. CPC A61M 25/0017; A61M 3/0283; A61M 2039/1077; A61M 3/0233; A61M 2210/1085; A61M 39/105

(57) ABSTRACT

An adaptor for use in continuous bladder irrigation, comprising a first hollow tube portion having at a first end a first connector portion adapted to be connected to a drainage outlet of a catheter wherein the first hollow tube portion being joined to a hollow inlet tube and a hollow outlet tube and the inlet tube having a fluid inlet connector and an insert portion extending from the fluid inlet connector and through the first hollow tube portion.

### FIELD OF THE INVENTION

The present invention is directed to an apparatus and method for providing continuous bladder irrigation in combination with a traditional Foley catheter.

### BACKGROUND OF THE INVENTION

A Foley catheter is a medical device formed of a flexible tube that is passed through the urethra and into the bladder to drain urine. Foley catheters are a common type of indwelling urinary catheter. The tube of a Foley catheter has two separated channels, or lumens, running down its length. In use, one of the lumens is open at both ends and drains urine into a collection bag. The other lumen has a valve on the outside end and connects to a balloon at the tip. When the catheter is inserted into the bladder, the balloon is inflated with sterile water so that the balloon expands inside the bladder to retain the catheter in place. Foley catheters are commonly constructed of silicone rubber. Use of Foley catheters increases the risk of catheter-associated urinary tract infection and other adverse effects.

Irrigation of the bladder is frequently necessary following surgery and is sometimes required because of other conditions, such as bleeding following an injury or due to stones or tumors, or in cases of bladder dysfunction or chronic obstruction. In one conventional procedure, a catheter is inserted into the bladder and remains in place in the patient as long as irrigation is needed. The catheter is connected to a drain bag or container between treatments to allow urine to be discharged. When an irrigation treatment is to be given, the catheter is disconnected from the drain bag and a plunger-type syringe is filled with sterile water, or other fluid suitable for irrigation, and connected to the catheter. The bladder is then irrigated by depressing the plunger of the syringe after which the plunger is retracted to withdraw the fluid from the bladder. The catheter is then disconnected from the syringe and reconnected to the drain bag until time for the next treatment. This frequent opening and closing of the system provides many opportunities for infection.

Continuous bladder irrigation is also frequently used to prevent the formation of blood clots after surgery as well as to remove urine. Continuous bladder irrigation involves continuous infusion of a sterile solution into the bladder, usually by using a three-way irrigation closed system with a triple-lumen catheter. One lumen is used to drain urine; another is used to inflate the catheter balloon, and the final lumen carries the irrigation solution. However, the insertion of a triple lumen catheter first usually requires the removal of the standard double lumen catheter. Replacing catheters always carries a risk of infection.

Therefore it can be seen that there is a need for a means and method of providing continuous bladder irrigation without replacing the standard double lumen Foley catheter and therefore reducing the risks of infection.

### SUMMARY AND OBJECTS OF THE INVENTION

The present invention provides an adaptor for use in continuous bladder irrigation, comprising a first hollow tube portion having at a first end a first connector portion adapted to be connected to a drainage outlet of a catheter wherein the first hollow tube portion being joined to a hollow inlet tube and a hollow outlet tube and the inlet tube having a fluid inlet connector and an insert portion extending from the fluid inlet connector and through the first hollow tube portion.

The present invention further provides a method of providing continuous bladder irrigation comprising the steps of attaching an inlet tube of an irrigation adaptor to a drainage port of an irrigant bag and removing air from an insert tube connected to the inlet tube by allowing irrigant liquid to flow from the irrigant bag through the insert tube so that air is removed from the insert tube and then stopping the flow of irrigant liquid and inserting the insert tube into a drainage lumen of an indwelling catheter and connecting the irrigation adaptor to a drainage port of the indwelling catheter, and connecting an outlet tube of the irrigation adaptor to a drainage bag and restarting the flow of irrigant liquid so that irrigant liquid flows through the insert tube.

The present invention also provides a medical device for providing continuous irrigation of an internal body part comprising a three-way adaptor formed of hollow tubing having a first opening, a second open and a third opening, the first opening being adapted for receiving a liquid and the second opening being adapted for connection to a catheter and the first opening having a hollow insert portion extending from the first opening and through the second opening and the hollow insert portion having a fluid outlet and wherein the second opening is larger than the hollow insert portion so that liquid may flow around the hollow insert portion through the second opening to the third opening.

Accordingly, it is an object of the invention to provide a device for facilitating continuous irrigation through a standard catheter;

Another object of the invention is to provide an adaptor to create a third lumen in a standard catheter;

Still another object of the invention is to provide a device which can be used to provide continuous irrigation and removed without removing a standard catheter.

According to the present invention, an adaptor for a catheter is provided for facilitating continuous irrigation.

It must be understood that no one embodiment of the present invention need include all of the aforementioned objects of the present invention. Rather, a given embodiment may include one or none of the aforementioned objects. Accordingly, these objects are not to be used to limit the scope of the claims of the present invention. The invention will become more fully apparent from the claims and the description in connection with the drawings.

### BRIEF DESCRIPTION OF THE DRAWINGS

FIG. 1 is plan view of the continuous bladder irrigation adaptor;

FIG. 2 is a fragmented view of the continuous bladder irrigation adaptor of FIG. 1 connected to a catheter;

FIG. 3 is an alternative embodiment of the connectors; and

FIG. 4 is a cross sectional view taken along lines 4-4 of FIG. 2.

### DETAILED DESCRIPTION OF THE PREFERRED EMBODIMENTS

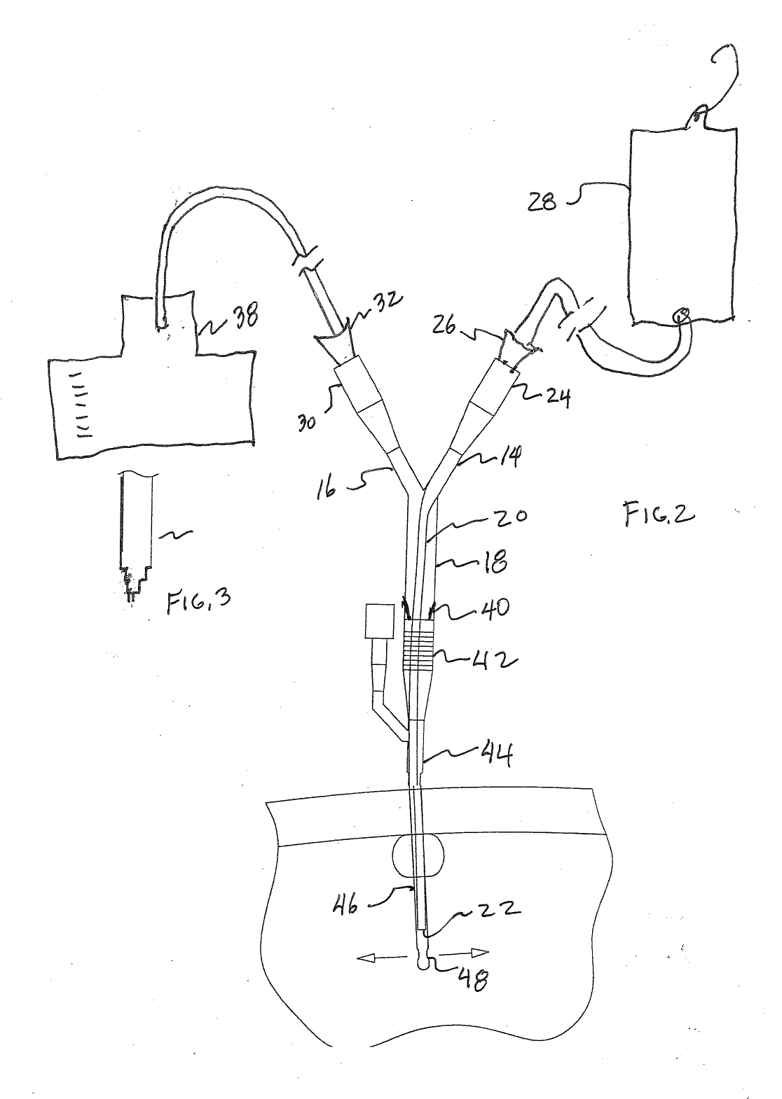

The adaptor 10 is shown in FIG. 1 and FIG. 2 includes a first Y-shaped portion 12 preferably formed of flexible material such as silicone or rubber having an inlet tube 14 and an outlet tube 16 and a connector 18. The connector 18 and the outlet tube 16 are hollow to allow drainage to flow through the connector 18 into the outlet tube 16. The inlet tube 14 includes an insert 20 of narrower diameter than the inside diameter of the connector 18 and the insert 20 extends through the connector 18. The insert 20 is a flexible hollow tube and may be formed of the same material as the Y-shaped portion 12, but is preferably less flexible than the material forming the Y-shaped portion. The insert 20 includes an insert outlet 22.

It should be understood that although a true Y shape is shown, the inlet tube 14 and the outlet tube 16 as well as the connector 18 can be of different lengths, i.e. the inlet tube 14 could be shorter or longer than the outlet tube 16 and both the inlet tube 14 and outlet tube could be of different lengths than the connector 18.

The inlet tube 14 includes an inlet port 24 for connecting to the drainage port 26 of an irrigant bag 28. The insert 20 may be constructed as one piece with the inlet port 24 and the inlet port 24 can be attached directly to or formed as part of the connector 18. The insert 20 can be attached to the inlet port 24 by welding, adhesive or a friction fit as long as a secure fluid tight seal is achieved. The inlet tube 14 and the inlet port 24 may be a single unitary part or may be formed of separate parts joined together by welding, adhesive or friction fit. The outlet tube 16 includes an outlet port 30 for connecting the outlet tube 16 to the tubing port 32 of a drainage bag 38. The connector 18 includes a connector port 40 for connecting the adaptor 10 to a urine drainage port 42 of a Foley catheter 44. Preferably, the connecting parts and tubing of the adaptor 10 are designed for use with standard medical equipment currently in use, however it is foreseeable that new standards may be developed for tubing materials, sizes and connectors and therefore this description of the preferred embodiment should not be construed as limiting the scope of the claims to the preferred embodiment.

In use, the drainage port 26 of an irrigant bag 28 is preferably attached to the inlet port 24 and fluid is allowed to flow through insert 20 to remove air. The insert 20 is then placed into the drainage lumen 46 of the indwelling Foley catheter 44 and the insert 20 preferably extends substantially the length of the drainage lumen 46 so that the insert outlet 22 is near the input-output ports 48 of the Foley catheter 44. The connector port 40 is then connected in a fluid-tight manner to the urine drainage port 42 of the Foley catheter 44. Outlet port 30 is connected to the tubing port 32 of the drainage bag 38. The irrigant fluid is then allowed to flow through the insert 20 to the input-output ports 48 of the catheter 44 and drainage flows through the drainage lumen 46 of the Foley catheter 44 to the outlet tube 16 to the drainage bag 38.

In the embodiment of FIG. 1 and FIG. 2, the inlet port 24 and outlet port 30 have smooth interior surfaces 54 and 56, respectively to allow a frictional engagement. Connector port 40 has a smooth exterior surface 58 for engaging the urine drainage port 42 of the catheter 44.

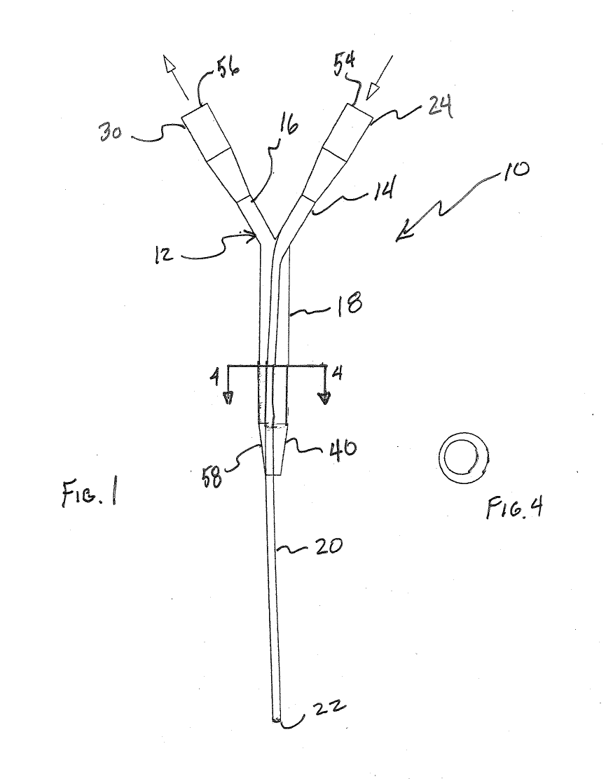

FIG. 3 illustrates an alternative stepped connector arraignment for the for the inlet port 22, the outlet port 24 and the connector port 26 wherein the ports have stepped connectors. It should be understood that the mode of connecting the ports 22, 24 and 26 could be either male or female connections and any of the connectors could be smooth or stepped or have a threaded or locking connection.

FIG. 4 is a cross-sectional view taken along lines 4-4 of FIG. 1 showing the connector 18 and the insert 20.

While the invention has been described with reference to a preferred embodiment thereof, it is to be understood that modifications or variations may be easily made without departing from the scope of the present invention which is defined by the appended claims.

1. An adaptor for use in continuous bladder irrigation, comprising:
a) a first hollow tube portion having at a first end a first connector portion adapted to be connected to a drainage outlet of a catheter;
b) said first hollow tube portion being joined to a hollow inlet tube and a hollow outlet tube; and,
c) said inlet tube having a fluid inlet connector and an insert portion extending from said fluid inlet connector and through said first hollow tube portion.

2. The adaptor for use in continuous bladder irrigation as set forth in claim 1, wherein said first hollow tube portion and said hollow inlet tube and said hollow outlet tube are formed of first flexible material and said insert portion is formed of a second flexible material.

3. The adaptor for use in continuous bladder irrigation as set forth in claim 2, wherein said first flexible material is more flexible than said second flexible material.

4. The adaptor for use in continuous bladder irrigation as set forth in claim 1, wherein said hollow outlet tube includes an outlet tube connector for connecting said hollow outlet tube to a tubing port of a drainage bag.

5. A method of providing continuous bladder irrigation, comprising the steps of:
a) attaching an inlet tube of an irrigation adaptor to a drainage port of an irrigant bag;
b) removing air from an insert tube connected to said inlet tube by allowing irrigant liquid to flow from the irrigant bag through said insert tube so that air is removed from said insert tube and then stopping the flow of irrigant liquid;
c) inserting said insert tube into a drainage lumen of an indwelling catheter and connecting said irrigation adaptor to a drainage port of said indwelling catheter;
d) connecting an outlet tube of said irrigation adaptor to a drainage bag; and,
e) restarting the flow of irrigant liquid so that irrigant liquid flows through said insert tube.

6. A medical device for providing continuous irrigation of an internal body part, comprising:
a) a three-way adaptor formed of hollow tubing having a first opening, a second open and a third opening;
b) said first opening being adapted for receiving a liquid;
c) said second opening being adapted for connection to a catheter;
d) said first opening having a hollow insert portion extending from said first opening and through said second opening;
e) said hollow insert portion having a fluid outlet; and,
f) wherein said second opening is larger than said hollow insert portion so that liquid may flow around said hollow insert portion through said second opening to said third opening.

7. The medical device as set forth in claim 6, wherein said hollow insert portion extends through said second opening into a drainage lumen of the catheter.

8. The medical device as set forth in claim 7, wherein said third opening is connected to a drainage bag for receiving drainage liquid from said catheter.

9. The medical device as set forth in claim 6, wherein said hollow tubing is formed of silicone.

10. The medical device as set forth in claim 6, wherein said three-way adaptor is connectable to a Foley catheter.
